(19) United States
(12) Patent Application Publication
(10) Pub. No.: US 2022/0387724 A1
(43) Pub. Date: Dec. 8, 2022

(54) Haptic Feedback of an Electronic Medical Device

(71) Applicant: Sanofi, Paris (FR)

(72) Inventors: Michael Helmer, Frankfurt am Main (DE); Maurice Toporek, Frankfurt am Main (DE)

(21) Appl. No.: 17/772,650

(22) PCT Filed: Oct. 26, 2020
(86) PCT No.: PCT/EP2020/080003

§ 371 Date: Apr. 28, 2022

(30) Foreign Application Priority Data
Oct. 28, 2019 (EP) 19306395.5

(51) Int. Cl. A61M 5/315

(52) U.S. Cl. CPC A61M 5/31578; A61M 2205/582; A61M 2205/02

(57) ABSTRACT

Implementations of the present disclosure are directed to a medical handheld device that includes an actuating feature configured to generate a trigger signal, a sensor configured to detect a functionality of the medical handheld device in response to the trigger signal, and a haptic source configured to generate a haptic signal including information associated with the functionality of the medical handheld device.

### CROSS REFERENCE TO RELATED APPLICATIONS

The present application is the national stage entry of International Patent Application No. PCT/EP2020/080003, filed on Oct. 26, 2020, and claims priority to Application No. EP 19306395.5, filed on Oct. 28, 2019, the disclosures of which are incorporated herein by reference.

### BACKGROUND

Many electronic medical devices, such as injection devices, enable patients to safely monitor physiologic parameters and receive treatment, without the need for constant supervision by medical staff. Often, electronic medical devices include a display that is configured to provide information about one or more functions or characteristics of the electronic medical devices. However, during the use of the electronic medical device, the user's visual focus could be away from the display of the electronic medical device. For example, the user could be using an electronic injection device and focus the view on the injection spot during the injection. Additionally, some users are visually impaired, which limits their ability to use the visual display of the electronic medical device.

### SUMMARY

Implementations of the present disclosure include handheld medical devices configured for providing haptic feedback related to a feature and/or use of the medical device. In accordance with one aspect of the present disclosure, a medical handheld device includes an actuating feature configured to generate a trigger signal, a sensor configured to detect a functionality of the medical handheld device in response to the trigger signal, and a haptic source configured to generate a haptic signal including information associated with the functionality of the medical handheld device.

In some implementations, the haptic source includes a vibrating element. The vibrating element includes at least one of a vibrating motor, a vibrating battery, and a speaker with vibrating membranes. The haptic source is enclosed within the medical handheld device. The haptic source is fixed at a location configured to enhance transmission of the haptic signal. The haptic source is integrated into an activation button. The medical handheld device can include a second sensor configured to detect a physiologic parameter of a patient. The actuating feature is configured to generate the trigger signal in response to detecting the physiologic parameter. The medical handheld device can include: a medicament reservoir configured to store a medicament to be expelled by the medical handheld device. The haptic signal includes information associated with expelling the medicament stored in the medicament reservoir. The vibrating element is included in an electric drive. At least a portion of the electric drive is connected to a plunger rod that is configured to be displaced to expel the medicament. The haptic source is integrated into at least one of an injection button, an input button, a dosage knob, and a dial grip. The medical handheld device can further include a motor including a first shaft configured to expel the medicament and a second shaft configured to generate the haptic signal.

In accordance with another aspect of the present disclosure, a medical system includes: a medical handheld device and an external device including a component monitored by the medical handheld device.

It is appreciated that systems in accordance with the present disclosure can include any combination of the aspects and features described herein. That is to say that methods in accordance with the present disclosure are not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF THE FIGURES

FIGS. 1A and 1B are exploded views of examples of medical devices in accordance with the present disclosure.

FIG. 2 illustrates another example of a medical device in accordance with the present disclosure.

FIG. 3 is a perspective view of a supplementary device to be releasably attached to the medical devices of FIGS. 1A and 1B in accordance with the present disclosure.

FIG. 4 is a perspective view of another example of a medical device in accordance with the present disclosure.

FIG. 5 is a flowchart illustrating example processes that can be executed to detect and transmit injection device-level data.

Like reference symbols in the various drawings indicate like elements.

### DETAILED DESCRIPTION

Implementations of the present disclosure are generally directed to generation of a haptic (vibrational) signal associated with an operation of a medical handheld device. More particularly, implementations of the present disclosure are directed to generation and transmission of haptic signals to indicate a condition of an electronic medical device to a user of the medical device. The condition can include a critical condition of the medical device, such as a detection of an abnormality associated with a function, a parameter, an operation or a mechanism (for example, a blocked needle, injection mechanism blocked, and out of range temperature). The haptic signal can also indicate non-critical conditions, such as operational conditions (for example, end of injection, end of a waiting time, and a holding time).

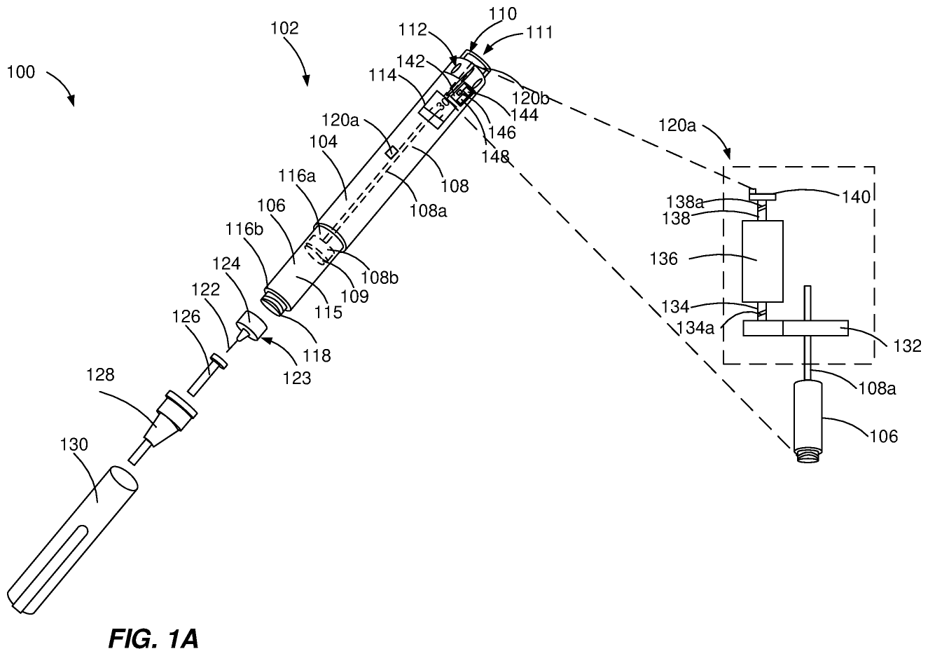

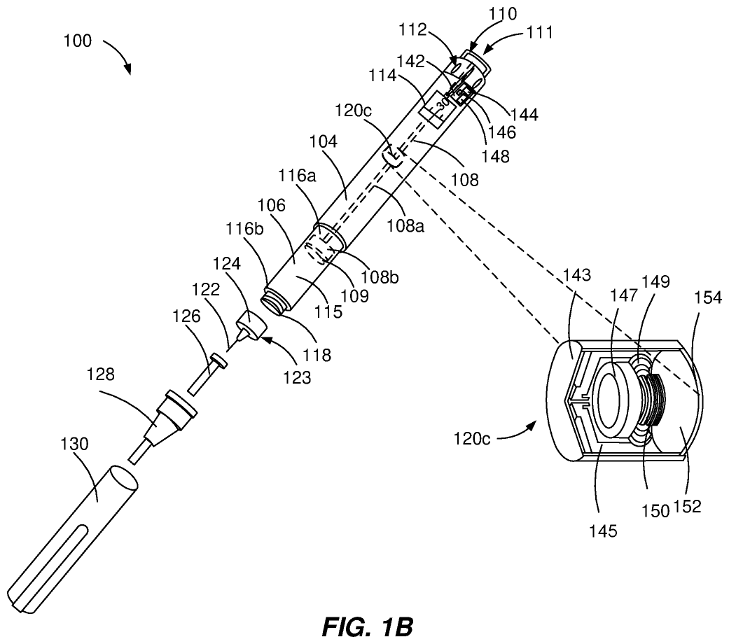

Electronic medical devices can be configured to provide haptic feedback in response to an analysis of critical parameters that can be detected by sensors of the electronic medical devices. For example, the haptic feedback can be provided to correct medical treatments and prevent usage errors. In some implementations, the haptic feedback can be a discrete signal that substitutes acoustical feedback during usage of the electronic medical devices in public environments. The electronic medical devices can be configured to maximize the transmission of the haptic feedback to the users of the electronic medical devices. The haptic feedback can be generated by haptic elements that can be included in any type of handheld medical devices, such as (but not limited to) the devices described with reference to FIGS. 1-4. The haptic elements can include any systems that can generate haptic signals, such as (but not limited to) the implementations described with reference to FIGS. 1-4.

FIGS. 1A and 1B illustrate a system 100 configured to provide a haptic feedback in accordance with one implementation of the present disclosure. The system 100 includes an exploded view of an example injection device (medical handheld device) 102. The injection device 102 includes one or more haptic elements 120a, 120b that can be used to provide feedback related to functions or use of the injection device 102. In some examples, the injection device 102 can be a pen device using replaceable medicament reservoirs (cartridges) 106. The pen device can be a pre-filled, injection pen or a reusable injection pen. The injection device 102 includes a housing 104, a medicament reservoir 106 (for example, medicament container or cartridge), a plunger 108, an injection button 110, a dosage knob 112, and a dosage window 114.

The housing 104 can be configured to define a medicament container or contain a reservoir 106 that can store an amount of medicament. The geometry and composition materials can be configured to optimize haptic signal transmission. For example, the reservoir 106 can include COC materials or glass to provide high purity, high moisture barrier, excellent haptic signal transmission, breakage prevention, and low density.

The reservoir 106 (for example, container) can be configured to contain a fluid medicament. The medicament can include a pharmaceutical formulation containing at least one pharmaceutically active compound. The medicament can include insulin analogs, insulin derivatives, analgesics, hormones, beta agonists, corticosteroids, or a combination of any of the above-mentioned drugs.

The plunger 108 can be configured to expel a portion of the medicament contained within the reservoir 106. The plunger 108 can include a plunger rod 108a and a plunger head 108b configured to push a stopper 109. The stopper 109 can be configured to expel a portion of a medicament stored within the reservoir 106 by moving within a tubular wall 115 of the reservoir 106 in a direction from the distal end 116a to the proximal end 116b, such that a position of the stopper 109 is indicative of an amount of the medicament within the reservoir 106. The terms “distal,” “distally” and “distal end” refer to the end of an injection device towards which a needle is provided. The terms “proximal,” “proximally” and “proximal end” refer to the opposite end of the injection device, towards which an injection button or dosage knob is provided. A large portion (for example, at least 90%) of the surface of the stopper 109 that is in contact with the medicament can be configured to be flat to minimize the dead filling volume of the injection device 102. A position of the stopper 109 can be associated with an amount of the medicament within the injection device 102.

The injection device can include an electric drive 111 for automatically expelling the medicament in response to activation of the injection button 110. The injection button 110 is configured to enable an injection of the medicament into the patient's body and to generate a trigger signal. The dosage knob 112 is configured to enable a selection of a dose of the medicament to be ejected from the injection device 102 and to generate a trigger signal. For example, a particular dose (volume) of the contained medicament can be set to be ejected from the injection device 102 by turning the dosage knob 112. Turning the dosage knob 112 can cause a haptic element 120a to provide haptic feedback to a user. The dosage window 114 is configured to display the selected dose. The selected dose can be displayed in multiples of so-called International Units (IU). One IU is the biological equivalent of about 45.5 micrograms of pure crystalline medicament (1/22 mg). An example of a selected dose displayed in dosage window 114 can for instance be 30 IUs, as shown in FIGS. 1A and 1B. The numbers displayed in dosage window 114 can be printed on a sleeve that is contained in housing 104 and mechanically interacts with a plunger 108 in reservoir 106. In some implementations, the dosage window 114 is an electronic display configured to display multiple information related to the injection device 102, including any of available amount of medicament, selected dose, ejected dose, battery life, expiration date of medicament, storage temperature and previously stored data.

The threading interface (nosepiece) 118 of the housing 104 can be attached to a needle hub 123. The needle hub 123 includes a needle 122 and a handle 124. The needle 122 is protected by an inner needle cap 126 and an outer needle cap 128, which in turn can be covered by a cap 130. When needle 122 can be inserted into a skin portion of a patient, and then injection button 110 is pushed, the medicament dose displayed in display window 114 is ejected from the injection device 102 and at least one of the haptic elements 120a, 120b, 120c generates a haptic signal.

The haptic signal can be characterized by a particular haptic pattern. Haptic patterns include a combination of signal durations at particular signal frequencies and pressures. Haptic patterns can include intermittent vibrations, constant vibrations, and vibration patterns. The vibration patterns can include: (i) long vibrations (for example, about few seconds) with short humanly perceptible pauses (for example, about few milliseconds), (ii) short vibrations (for example, less than about 1 second) with long pause (for example, about few seconds), (iii) pauses with increasing durations, (iv) pauses with decreasing durations, (v) single/double/triple short vibrations; (vi) soft (for example, with an amplitude from about 10 to about 50 micrometers) vibration, (vii) strong (for example, with an amplitude from about 100 to about 500 micrometers) vibration. In some implementations, short (for example, less than about 3 seconds) haptic signals with simple haptic patterns (for example, with a single constant frequency that is below approximately 220 Hz or a pair of low and high frequencies) can be used to provide positive feedback. In some implementations, long (for example, longer than about 3 seconds) haptic signals with simple haptic patterns (for example, with a single constant frequency or a pair of low and high frequencies) or complex haptic patterns (for example, a combination of multiple frequencies or strong signals with high frequencies) can be used to provide an indication of a critical condition. Long vibrations or pulses are typically in the range of about 0.1 Hz to about 5 Hz, preferably from about 1 Hz to about 3 Hz. Short vibrations or pulses are typically in the range of about 5 Hz to about 30 Hz, preferably from about 5 Hz to about 12 Hz. Similar intervals hold true for short and long pauses, respectively.

For example, when the needle 122 of injection device 102 remains in contact with a portion of the skin for a particular period of time (for example, 10 seconds) after the injection button 110 is pushed, a high percentage of the dose (for example, more than 98%) is actually injected into the patient's body. In response to completing the ejection of the medicament at least one of the haptic elements 120a, 120b, 120c can generate an additional haptic signal, which can be different (for example, can have a different haptic pattern) from the haptic signal produced before the ejection of the medicament. The injection device 102 can be used for several injection processes until either medicament reservoir 106 is empty or the expiration date of injection device 102 (for example, 28 days after the first use) is reached. Before using injection device 102 for the first time, it can be necessary to perform a so-called “prime shot” to remove air from medicament reservoir 106 and needle 122, for instance by selecting two units of medicament and pressing injection button 110 while holding injection device 102 with the needle 122 upwards.

The haptic elements 120a, 120b (illustrated in FIG. 1A), 120c (illustrated in FIG. 1B) can be configured to generate a haptic signal in response to receiving a trigger signal. The haptic elements 120a, 120b, 120c can be included in any part of the injection device 102. The haptic elements 120a, 120b, 120c can be fixed at a location that enables maximum transmission of the haptic signals through the housing 104, such as proximal to the injection button 110 or around a middle section of housing 104 proximal to the wall of the housing 104. In some implementations, the haptic elements 120a, 120b, 120c are in direct contact with the housing 104 to maximize the transmission of haptic signals through the housing 104. In some implementations, the composition, geometry, and structure of the housing 104 can be configured such that the haptic signals are maximally perceived near the haptic elements 120a, 120b, 120c and are perceivable throughout the entire surface of the housing 104. For example, the configuration of the housing 104 and the haptic elements 120a, 120b, 120c can enable transmission of the haptic signal to a user holding the injection device 102, independent of the holding configuration.

As illustrated in FIG. 1A, the haptic elements 120a, 120b can include a piston rod 132, a shaft 134 with clutch 134a for a clockwise rotation, a motor 136, a shaft 138 with clutch 138a for an anti-clockwise rotation, and an unbalanced disk 140. The shaft 134 is configured for driving the medicament drive train. The shaft 138 is configured for generating haptic signals. For example, the piston rod 132 can be attached to the plunger rod 108a such that the haptic signals can be associated to a movement of the plunger rod 108a. The haptic signal is generated in response to a trigger signal by the unbalanced disk 140 as activated by the motor 136 and transmitted by the shaft 138. The trigger signal can include a mechanical signal (for example, movement of the plunger rod 108a) or an electrical signal (for example, detection that a battery life of the injection device 102 is at a critical level). The haptic elements 120a, 120b, 120c can include one or more clutches 138a (for example, drive plates) to disengage the unbalanced disk 140 when the injection device 102 injects a dose (by clockwise rotation) and engage the unbalanced disk 140 by anti-clockwise rotation. The clutch 138a can be a movable (for example, anti-clockwise rotational) or a stationary clutch. The anti-clockwise clutch 138a can have a centrifugal clutch unit to enable the piston rod 132 to be withdrawn slowly (when the cartridge is changed) without generating a haptic (vibration) signal. The haptic signal can be produced by the rotation of the motor 136 that activates a fast anti-clockwise rotation of the shaft 138.

As illustrated in FIG. 1B, the haptic element 120c can be configured to generate haptic signals for a user of the device 102. The haptic element 120c can include a vibrating battery or a vibrating drive train. The haptic element 120c includes a housing 143 configured to cover the internal elements of the haptic element 120c and a basket 145 configured to support the internal elements of the haptic element 120c. In some implementations, the haptic element 120c can be a speaker with vibrating membranes, as illustrated in FIG. 1B. The vibrating membranes can include a material having a particular elastic modulus that enables vibrations at a frequency and amplitude optimal for transmission through the housing 104. For example, the vibrating membranes can include a polymer thin film. The haptic element 120c can include a magnet 147, a spider 149, a voice coil 150, a diaphragm 152, and a suspension 154.

In some implementations, the injection device 102 can include a detection module 142. The detection module 142 can be configured to determine the functionality of the injection device 102 and a medicament amount stored within the medicament reservoir 106. For example, the detection module 142 is configured to monitor and identify critical values of the amount of medicament that is contained within the injection device 102 or set to be delivered by the injection device 102.

The detection module 142 can include a power source 144, a sensor 146, and a processor 148. The power source 144 can be an integrated battery or a super capacitor. In some implementations, the power source 144 can include an energy harvester configured to harvest energy from interrogation signals emitted by an external device or mechanical energy generated by an interaction of a user with the injection device 102. The power source 144 can be configured to supply energy to the components of the detection module 142 continuously or under particular conditions (for example, in response to activation of the injection button 110).

The sensor 146 can include multiple sensors. The sensor 146 can include mechanical, electrical, optical, acoustical sensors or a combination thereof. The sensor 146 can be configured to detect values associated to multiple features of the injection device 102. For example, the sensor 146 can be configured to detect the status of the power source 144, the position of the stopper 109 for each injection, and the amount of medication that is remaining in the medicament reservoir 106. For example, the sensor 146 can be configured to detect the status of the power source 144 (for example, low power of the power source 144), the position of the stopper 109 for each injection (for example, the position of the stopper 109 relative to a particular stopper position), low or high temperature (for example, temperature outside a particular temperature interval such as 0° C. to 15° C.), operation failure, and the amount of medication that is remaining in the injection device 102. Examples of operation failures detectable by the sensor 146 include premature end of injection due to stalling and/or clogging of the needle 122, cap removal pending, direction of device is incorrect (for example, the device longitudinal axis forms an angle of less than 80 degrees with a skin of the patient). Another situation could be associated with an amount of medicament in the medicament reservoir 106 (for example, empty or insufficient medicament for an injection). The amount of medicament can be compared to a dose set by the user of the injection device 102 (for example, user selects 23 units to be injected but only 16 units are left in the container) or to a statistically determined dosage (for example, average of injected doses within a predetermined time interval). The electronics in the injection device 102 can be configured to keep track of medicament dose history and thus have information available on the current amount of medicament in the container.

The processor 148 can be configured to determine the amount of the medicament within the injection device based at least in part on the electrical signal and transmit the data including the amount of the medicament to the display 114. The processor 148 can be configured to compare the values detected by the sensor 146 to corresponding threshold or reference values to generate a trigger signal in response to identifying critical operations or unusual conditions. The processor 148 can be configured to transmit the trigger signal to haptic one or more elements 120a, 120b, 120c to generate a haptic signal to alert a user of the injection device 102 of the critical condition. In some examples, the processor 148 includes a controller configured to correct a critical condition (for example, shift the position of the stopper 109 to a standard predetermined position) and to generate an additional trigger signal to indicate the correction of the critical condition. The processor 148 can be configured to determine information/data and generate trigger signals throughout the lifetime of the medicament reservoir 106 and/or the injection device 102.

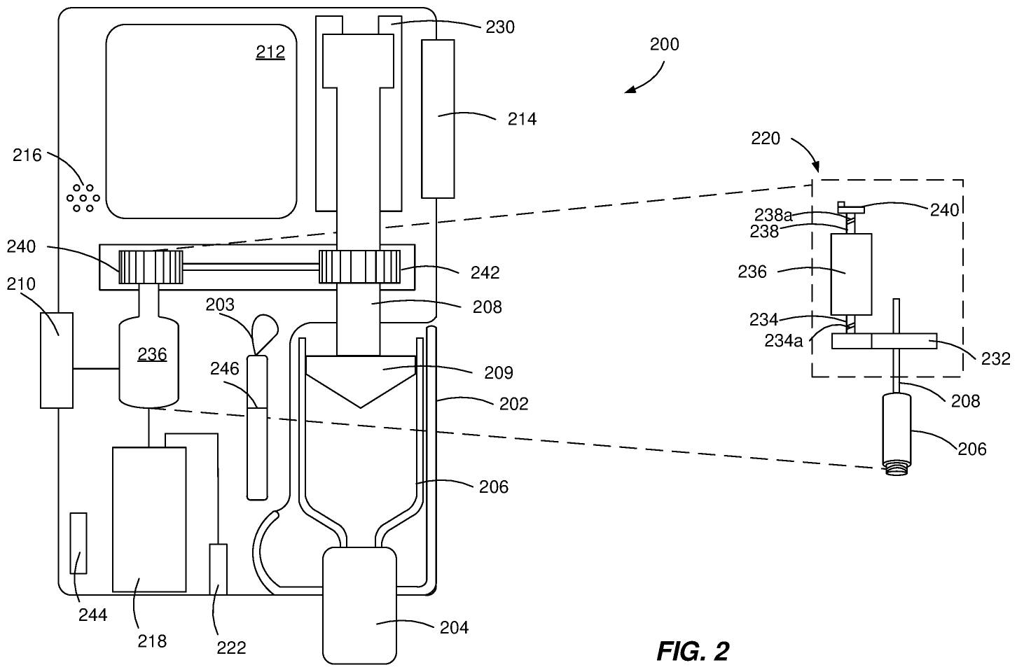

FIG. 2 illustrates an example of an injection device 200 configured to provide a haptic feedback in accordance with one implementation of the present disclosure. The injection device 200 includes a medicament cartridge holder 202, a needle hub/tip 204, a medicament reservoir 206 (for example, medicament cartridge), a plunger 208, an injection button 210, a display 212, a control panel 214, speaker 216, a battery 218, a haptic element 220, a charging port 222.

The medicament cartridge holder 202 is configured to retain a medicament cartridge 206. In some implementations, the cartridge holder 202 may be retained in place by a latch 203. The injection device 200 includes an expulsion mechanism configured to eject a portion of the medicament from the medicament cartridge 206 via a needle hub/tip 204 in response to an activation signal. The medicament cartridge 206 is an expendable and replaceable part of the injection device 200. The medicament cartridge 206 can be inserted within and removed from the medicament cartridge holder 202. The medicament cartridge 206 contains a medicament.

The expulsion mechanism can include the plunger 208 and the stopper. The plunger 208 can be configured to expel a portion of the medicament contained within the medicament cartridge 206. The plunger 208 can include a plunger rod and a plunger head configured to push a stopper 209. The stopper 209 can be configured to expel a portion of a medicament stored within the medicament cartridge 206, such that a position of the stopper 209 is indicative of an amount of the medicament within the medicament cartridge 206. In some implementations, the plunger 208 is retained within dovetail guide slits 230 in the injection device 200. The expulsion mechanism includes a belt drive 242 to drive the plunger 208. The motor 236 drives the belt drive 242. Guidance of the piston rod in rotatory direction is provided via the housing using the dovetail guide slits 230.

The haptic element 220 can include a piston rod 232, a shaft 234 with clutch 234a for a clockwise rotation, a motor 236, a shaft 238 with clutch 238a for an anti-clockwise rotation, and an unbalanced disk 240. The shaft 234 is configured for driving the medicament drive train. The shaft 238 is configured for generating haptic signals. For example, the piston rod 232 can be attached to the plunger rod 208a such that the haptic signals can be associated to a movement of the plunger rod 208a. The haptic signal is generated in response to a trigger signal by the unbalanced disk 240 as activated by the motor 236 and transmitted by the shaft 238. The motor 236 may include an absolute rotation sensor and/or a stepper motor for controlling the number of rotations of the motor 236. The piston position of the plunger 208 relative to the medicament cartridge 206 can, in some implementations, be determined from the number of motor 236 revolutions. In some implementations, the absolute position of the plunger 208 is determined by the sensor 246 relative to a reference position measured after the medicament cartridge 206 is inserted. In some implementations, the scanner unit 246 determines a corresponding plunger stroke by measuring the medicament level in the medicament cartridge 206. The motor 236 can use the corresponding plunger stroke to provide a number of rotations that can provide that plunger stroke, such that the corresponding plunger position in the medicament cartridge 206 is reached. The motor 236 may also be provided with an absolute value for a distance between the plunger 208 and the stopper 209 before the injection device is first used. The plunger to stopper distance can ensure that the motor 236 displaces the plunger 208 by the correct distance to expel the selected amount of medicament, and help prevent incorrect amounts of medicament from being expelled.

The clutches 234a, 238a (for example, drive plates) can be configured to disengage the unbalanced disk 240 when the injection device 200 injects a dose (by clockwise rotation) and engage the unbalanced disk 240 by anti-clockwise rotation. The clutch 238a can be a movable (for example, anti-clockwise rotational) or a stationary clutch. The anti-clockwise clutch 238a can have a centrifugal clutch unit to enable the piston rod 232 to be withdrawn slowly (when the cartridge is changed) without generating a haptic (vibration) signal. The haptic signal can be produced by the rotation of the motor 236 that activates a fast anti-clockwise rotation of the shaft 238.

The injection button 210 can be used to provide an activation signal to initiate expulsion of medicament from the medicament cartridge 206 by the expulsion mechanism. In some implementations, the injection button 210 is attached to a haptic element 220 such that activating the injection button 210 can cause the haptic element 220 to provide haptic feedback to a user of the injection device 200.

The display 212 can include touch screen features. The display 212 can provide a visual indication of the status of the injection device 200, such as a mode the injection device 200 is in, a battery status, and/or whether a power supply is connected to the injection device 200. In some implementations, the display 212 is synchronized with the haptic element 220 such that each visual indication can correspond to a particular haptic signal with a distinct pattern. The display 212 can also be used to communicate messages to the user. The display 212 can include a Liquid Crystal Display (LCD) or Light Emitting Diode (LED) or OLED screen or similar. In some implementations, the display 212 may include touch screen functionality, allowing the display 212 to receive user inputs. The touch screen functionality can be used to support installation and troubleshooting procedures. Alternatively or additionally, the display 212 may be accompanied by one or more push buttons or keys in the vicinity of the display, allowing the user to interact with the injection device 200, for example by navigating a cursor through a collection of menus and options on the display 212.

The control panel 214 can include one or more of: a power switch (an on/off switch), a release switch for the cartridge holder 202, dose setting buttons, and controls for the display 212. In some implementations, the control panel 214 is attached to the haptic element 220 such that activating the control panel 214 can cause the haptic element 220 to provide haptic feedback to a user of the injection device 200.

The speaker 216 can be configured to transmit audio signals to the user of the injection device 200 in the form of sound. The injection device 200 can include a microphone (not shown) for receiving user inputs including sound signals. In some implementations, the sound signals can be used as trigger signals for the haptic element 220.

The injection device 200 can be powered by a battery 218. The battery 218 can include a rechargeable battery. A power port 222 can be included in the injection device 200 for charging the injection device 200. The power port 222 can, in some implementations, also be used to recharge the battery 218. In some implementations, the battery 218 and/or the power port 222 can be attached to the haptic element 220 such that a status of the battery 218 can cause the haptic element 220 to provide haptic feedback to a user of the injection device 200.

In some implementations, the injection device 200 can include a network interface 244 configured to connect the injection device 200 to a network. The network interface 244 can include one or more of: a Wi-Fi interface; an Ethernet interface; a Bluetooth transceiver; and/or a USB interface. The injection device 200 can connect to a local area network (LAN) and/or the Internet via the network interface 244, enabling the injection device 200 to exchange data with other devices connected to the network. The injection device 200 may also use the network interface 244 to connect directly with additional devices, using, for example, a Bluetooth connection. The injection device 200 can be connected to a data server via the network interface 244. Through this connection the apparatus can share data in a bi-directional manner with the data server. The data server can be remotely located from the injection device 200, for example at a data centre controlled by a medicine supplier or a device manufacturer. In some implementations, the data centre can be part of a distributed computer system. For example, the computer system may be a “cloud”-based system. In some implementations, the data server may be located locally to the apparatus on the same Local Area Network.

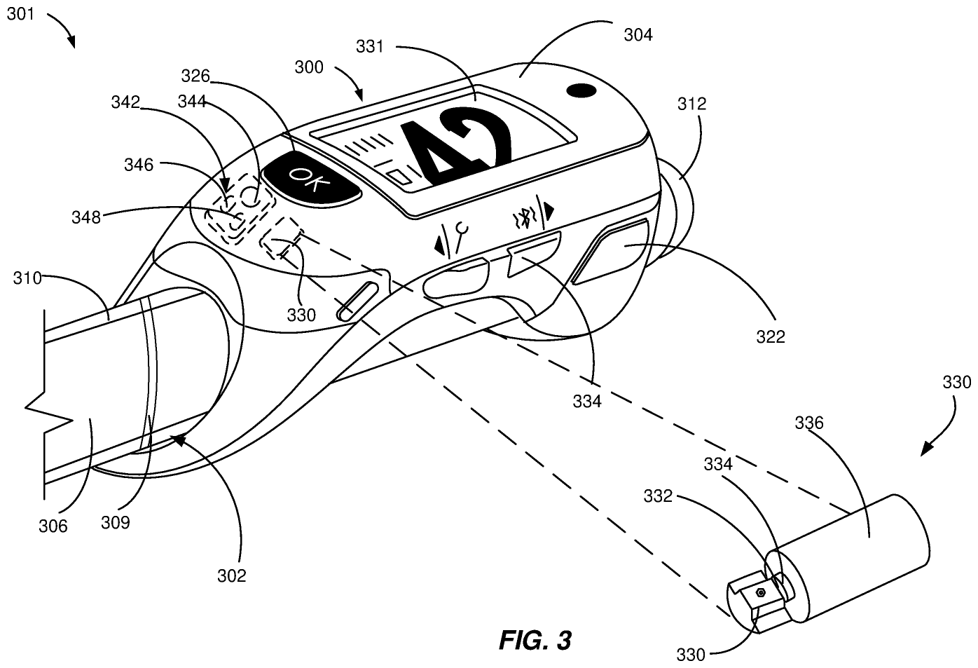

In some implementations, the injection device 200 can include a sensor 246. The sensor 246 can include an optical CCDM unit. The sensor 246 is configured to monitor optical codes on medicament cartridges 10 to register individual medicament cartridges 206. For example, the sensor 246 may record a medicament identifier of the medicament cartridge 206. In some implementations, the sensor 246 can be used to monitor one or more of: a medicament color and/or a medicament level in the medicament cartridge 206. In some implementations, the sensor 246 can be attached to the haptic element 220 such that a value measured by the sensor 246 can cause the haptic element 220 to provide haptic feedback to a user of the injection device 200. FIG. 3 is a schematic illustration of a medical system 301 including an example of a supplementary device (medical handheld device) 300 and an injection device 302. The supplementary device 300 can be configured to provide a haptic feedback in accordance with one implementation of the present disclosure. The supplementary device 300 can be releasably attached to an injection device 302 (for example, injection device 300 of FIG. 3). The supplementary device 300 includes a housing 304 with a mating unit configured to embrace the housing 310 of injection device 302 so that supplementary device 300 sits tightly on housing 310 of injection device 302. The housing 304 can be configured to enable removal of the supplementary device 300 from the injection device 302, for instance when injection device 302 is empty and has to be replaced.

Supplementary device 300 can include one or more haptic elements 320 and multiple (for example, three) user input buttons, illustrated schematically as a button 322. The haptic elements 320 can be fixed at a location that enables transmission of the haptic signals through the housing 310. For example, the haptic elements 320 can be located near to any of the buttons 322, 324, 326. The haptic element 320 can be configured to generate haptic signals for a user of the supplementary device 300. The haptic element 320 can be a microdrive vibrating motor. For example, the haptic element 320 can include an eccentric mass counter weight 330, a ball race bearing 332, a front cap 334, and a motor 336.

The buttons 322, 324, 326 can include mechanical switches or electrical coupling. The first button 322 can be an input button. The input button 322 can be configured to enable a user to turn on or off the supplementary device 300, to trigger actions (for instance to cause establishment of a connection to or a pairing with another device, and/or to trigger transmission of information from supplementary device 300 to another device), or to confirm an action. The second button 324 can be a communications button. The third button 326 can be a confirm button (for example, OK button).

The supplementary device 300 can include a detection module 342 for retrieving information from the injection device 302 (for example, amount of medicament in medicament reservoir 306). The display unit 321 of supplementary device 300 can be configured to display information acquired by the detection module 342. The dosage window of injection device 3 302 can be obstructed by supplementary device 300 when attached to injection device 302.

The detection module 342 can include a power source 344, a sensor 346, and a processor 348. The power source 344 can be an integrated battery or a super capacitor. In some implementations, the power source 344 can include an energy harvester configured to harvest energy from interrogation signals emitted by an external device or mechanical energy generated by an interaction of a user with the supplementary device 300. The power source 344 can be configured to supply energy to the components of the detection module 342 continuously or under particular conditions (for example, in response to activation of one of the buttons 312, 322, 324, 326) and to the haptic element 320.

The sensor 346 can be configured to detect values associated to multiple features of the injection device 302. The sensor 346 can include multiple sensors. The sensor 346 can include mechanical, electrical, optical, acoustical sensors or a combination thereof. For example, the sensor 346 can be configured to detect an amount of medicament in medicament reservoir 306, the status of the power source 344 (for example, low power of the power source 344), the position of the stopper 309 for each injection (for example, the position of the stopper 309 relative to a particular stopper position), low or high temperature (for example, temperature outside a particular temperature interval such as 0° C. to 35° C.), operation failure, and the amount of medication that is remaining in the injection device 302.

The processor 348 can be configured to compare the values detected by the sensor 346 to corresponding threshold or reference values to generate a trigger signal in response to identifying critical operations or unusual conditions (for example, battery life decreased to a minimal operational level). The processor 348 can be configured to transmit the trigger signal to haptic the haptic element 320 to generate a haptic signal to alert a user of the supplementary device 300 of the critical condition. In some examples, the processor 348 includes a controller configured to correct a critical condition (for example, prevent the delivery of a medicament under critical conditions) and to generate an additional trigger signal to indicate whether the critical condition was corrected (for example, battery was replaced or recharged). The processor 348 can be configured to determine information/data and generate trigger signals throughout the lifetime of the supplementary device 300 and/or the injection device 302.

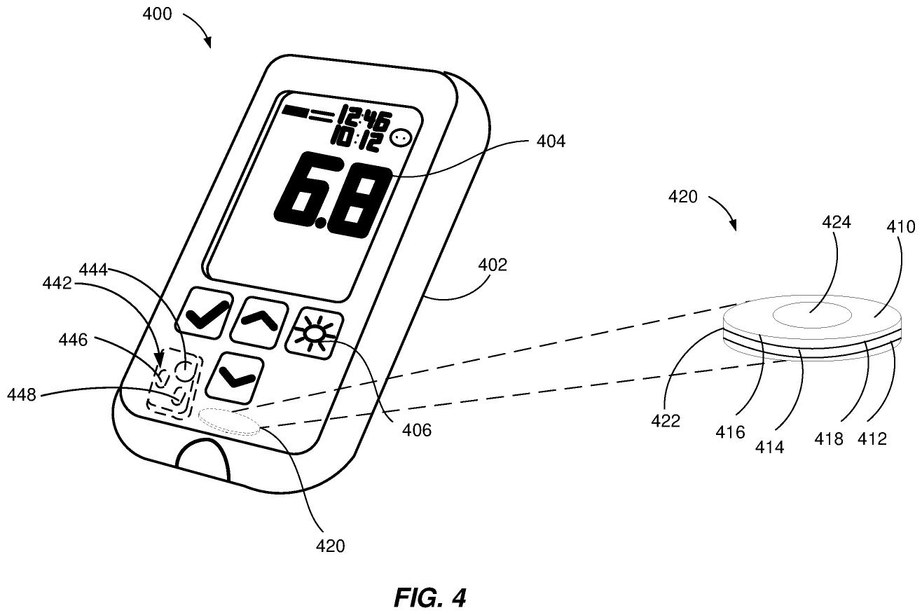

FIG. 4 shows a health monitor device (medical handheld device) 400 configured to provide a haptic feedback in accordance with one implementation of the present disclosure. The health monitor device 400 includes a housing 402, a display unit 404, a plurality of input buttons 406, a haptic element 420, and a detection module 442. The display unit 404 and the plurality of input buttons 406 are positioned to be accessible through the housing 402. The plurality of input buttons 406 are configured to allow the user of the health monitor device 400 to provide an input or enter data or relevant information associated with the operation of the health monitor device 400. For example, the user of the health monitor device 400 can operate the one or more input buttons 406 to enter a calibration code associated with a test strip, or other fluid sample reception means, for use in conjunction with the health monitor device 400.

In some implementations, the health monitor device 400 can include a blood glucose meter with bolus calculation function configured to calculate a single bolus dosage of a medication such as insulin such as long acting, fast acting or rapid acting insulin. Additionally, the user can operate the one or more input buttons 406 to adjust time and/or date information, as well as other features or settings associated with the operation of the health monitor device 400.

In some implementations, a strip port for receiving the test strip can be integrated with the housing 402 of the health monitor device 400, or alternatively, can be provided in a separate housing or as a separate component that can be physically or electrically coupled to the health monitoring device 400. For example, a component including the strip port can be provided in a separate snap on type housing which physically snaps onto the housing 402 of the health monitor device 400.

In some implementations, the health monitor device 400 can be configured to automatically enter into a medication dosage calculation mode to, for example, estimate a medication dosage amount based on information stored in the health monitor device 400 (for example, the patient's insulin sensitivity), and/or prompt the patient to provide additional information, such as the amount of carbohydrate to be ingested by the patient for determination of, for example, a carbohydrate bolus dosage determination. The patient can operate the input buttons 406 in conjunction with the user interface menu provided on the display unit 404 to provide additional user information.

In some implementations, the health monitor device 400 can be configured to prompt the patient to select whether to retrieve a predetermined or preprogrammed medication dosage amount such as, for example, a correction bolus or a carbohydrate bolus, following the display of the determined analyte level from the test strip. The health monitor device 400 can be configured to automatically prompt the user or patient to select whether a medication dosage determination is desired following an analyte testing using the test strip. In some implementations, the prompt is provided as a display on the display unit 404 and as a haptic signal generated by the haptic element 420.

The haptic element 420 can include a vibrating battery. The haptic element 420 can include a housing 410, a base member 412, one or more plates 414, one or more piezoelectric elements 416, a connection member 418, a vibration amplifying part 422, and a circuit board 424. The housing 410 can have an internal space and form the external appearance of the haptic element 420. For example, the housing 410 can have a coin shape, a flat rectangular shape or a thin curved shape, matching the curvature of the housing 402. The base member 412, the plate 414, the piezoelectric element 416, the connection member 418, the vibration amplifying part 420, and the circuit board 422, can be installed in the internal space of the housing 410.

In some implementations, the input of the user, one or more operations of the health monitor device 400 and one or more parameters measured by the health monitor device 400 are processed by the detection module 442. The detection module 442 can include a power source 444, a sensor 446, and a processor 448. The power source 444 can be an integrated battery or a super capacitor. In some implementations, the power source 444 can include an energy harvester configured to harvest energy from interrogation signals emitted by an external device or mechanical energy generated by an interaction of a user with the health monitor device 400. The power source 444 can be configured to supply energy to the components of the detection module 442 continuously or under particular conditions (for example, in response to activation of one of the buttons 406) and other components of the health monitor device 400.

The sensor 446 can be configured to detect values associated to multiple parameters of the health monitor device 400. The sensor 446 can include digital sensors. For example, the sensor 446 can be configured to detect the status of the power source 444, the results of analyte testing, and the amount of medication that is remaining in the health monitor device 400.

The processor 448 can be configured to compare the values detected by the sensor 446 to corresponding threshold or reference values to generate a trigger signal in response to identifying critical operations or unusual conditions. The processor 448 can be configured to transmit the trigger signal to activate the haptic element 420 to generate a haptic signal to alert a user of the health monitor device 400 of the critical condition. In some examples, the processor 448 includes a controller configured to generate an additional trigger signal to indicate the correction of the critical condition. The processor 448 can be configured to determine information/data and generate trigger signals throughout the lifetime of the health monitor device 400 and/or the health monitor device 400.

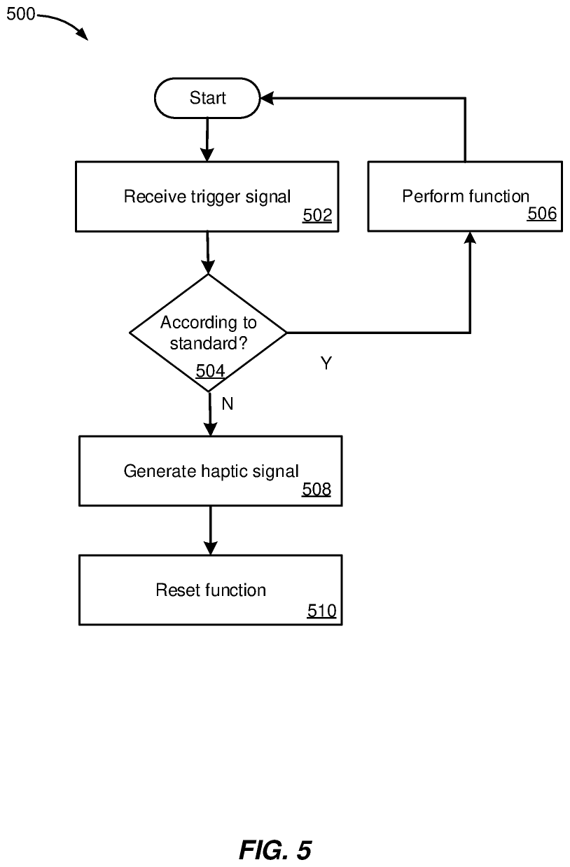

FIG. 5 is a flowchart illustrating an example processes 500 that can be executed to generate haptic signals to assist a functionality of a medical device. The process 500 can be executed by devices and systems described with reference to FIGS. 1-4.

The process 500, illustrated by FIG. 5, begins by receiving a trigger signal (502). The trigger signal can be generated by in response to receiving an input by an actuating feature (for example a button of a medical device) or by a sensor included in a handheld electronic medical device. The handheld electronic medical device can include an injection device, a supplementary device to be releasably attached to a medical device or a health monitor device (for example, a blood glucose monitor). In some implementations, the trigger signal can be generated in response to a critical physiologic parameter of a patient measured by the electronic medical device. In some implementations, the trigger signal can generated in response to a user input (for example, a priming operation or an input provided using a graphical user interface) on the handheld medical device. An example of a priming operation performed with an injection device can include selecting a particular number (for example, one or two) of units of medicament and pressing an injection button while holding the injection device with the needle upwards. Another example of a priming operation of the medical device can include pressing a priming button or an on/off button of the medical device configured as an electric switch. The trigger signal can include at least one of a mechanical signal and an electric signal. In some implementations, the trigger signal can include an on switch signal that is generated during a particular time period (for example, a given number of seconds or 1-2 minutes). The trigger signal can include a command to generate a verification of operational conditions and parameters of the medical device.

One or more operational conditions of the medical device, parameters of the medical device, and/or physiologic patient parameters are compared to standard values to differentiate between standard (normal) values and abnormal (critical) operations (504). Any conditions or features of the medical device or the medicament stored by the medical device that can affect what is defined as a standard injection of the expected amount of the medicament can be analyzed. The operational conditions or features of the medical device can include a battery lifetime of the medical device, an amount of the medicament to be ejected, an amount of the medicament available within the medicament container, a current date, a medicament identifier, and a medicament property.

The operational conditions or features of the medical device can be based on a measurement of an electric signal, a scan of a unique identifier for the injection device, an optical measurement of a property of the medicament (for example, a descent rate of a sediment within the medicament, density, optical absorption coefficients and/or temperature of the medicament), a temperature measurement (for example, a medicament temperature), and an internal clock measurement (for example, a timestamp of receipt of the ultrasound signal). The medicament temperature can be determined for medicaments including an additive or sediments based on the light signal detected by the receiver and known descent rate of the sediments. The amount of the medicament within the injection device can be determined based on one or more characteristics of the light signal detected by the receiver. For example, the position of an injection device stopper can be determined based on a phase of the reflected light signal using a particular detection method. The detection method can include an interferometric distance detection method and/or a phase modulation method combined with known geometrical characteristics (for example, area of cross-section) of the injection device and medicament reservoir. In some implementations, the amount of the medicament within the injection device can be determined based on differential measurements associated with an initial position of the plunger (prior to dispensing the medicament) and a final position of the plunger (after dispensing the medicament).

In some implementations, the comparison includes an evaluation relative to preset threshold values (for example, a minimum percentage of battery lifetime, a minimum number of days to an expiration date, a minimum amount of medicament in the medicament reservoir relative to a selected amount of medicament to be expelled and an expected amount of the medicament). The insertion of the correct drug in the injection device can be determined based on the light signal detected by the receiver and a comparison between determined optical absorption coefficients and known optical absorption coefficients of medicaments. The match between medicament volume and available medicament volume can be determined based on the light signal detected by the receiver and optical absorption coefficients of medicaments versus air. The match between medicament volume and available medicament volume can be used to confirm that the medicament reservoir is not empty or partially empty. The medicament temperature can be compared to threshold storage temperatures for medicaments including additives or sediments based on the light signal detected by the receiver and known descent rate of the sediments. The amount of the medicament selected by the user to be expelled by the injection device can be compared to the expected amount of the medicament (an average of the amount of the medicament expelled during an analyzed time interval) and to the amount of medicament stored in the medicament reservoir. If the amount of the medicament selected by the user to be expelled by the injection device is larger than the expected amount of the medicament (by a particular preselected percentage) or larger than the amount of medicament stored in the medicament reservoir, the comparison result indicates a critical condition.

In response to determining that the comparison result indicates a normal (noncritical) operation condition, a function of the medical device is performed (506). For example, the selected amount of medicament is expelled by the injection device. In response to determining that the comparison result indicates a critical (nonstandard) operation condition, a haptic signal is generated (508). The haptic signal can include a mechanical vibration transmitted through the housing of the medical device. The haptic signal can have a multiple characteristics corresponding to a type of the determined critical condition. The haptic signal can be characterized by a particular haptic pattern. Haptic patterns include a combination of particular frequencies signal durations at particular signal frequencies and pressures. Haptic patterns can include intermittent vibrations, constant vibrations, and vibration patterns. Examples of vibration patterns are included in Tables 1-3.

TABLE 1
Examples of vibration patterns used as feedback for
auto injector devices provided by electronic design.

| Action | Vibration Pattern |
|---|---|
| Start of injection | No action |
| End of injection | One short pulse |
| Holding time ended | Two short pulses |
| Operational user error | Three short pulses, three long pulses, |
| | three short pulses or alternatively |
| | continuous vibrations optionally short |
| | pauses. |
| Device error | Three short pulses, three long pulses, |
| | three short pulses (SOS) |
| “Reminder” function: user | “endless” vibration pattern, for example |
| interrupts injection procedure, | long pulse, long pause; |
| for example removes cap, puts | |
| pen aside to take phone call | |
| and then forgets | |

TABLE 2
Examples of vibration patterns used as feedback for standard
injection devices provided by electronic design.

| Action | Vibration Pattern |
|---|---|
| Dose Dialing | No action |
| Start of injection | No action |
| End of injection | One short pulse |
| Holding time ended | Two short pulses |
| Operational user error | Three short, three long, three short |
| | (SOS) or alternatively continuous |
| | vibrations optionally short pauses. |
| Device error | Three short, three long, three short |
| | (SOS) |
| Environmental condition a | Two long pulses with short pause in |
| User takes device from the | between; then long pause; for example, |
| fridge; device and | repeat 3 times. |
| medicament need to warm up | |
| before use; indication when | |
| temperature range is reached, | |
| Environmental condition b | Three short, three long, three short |
| Device including medicament | (SOS) or triplet of short pulses and |
| lies in the sun light, for | long pause; needs user confirmation |
| example, and gets warm | to indicate that the user has understood |
| | the alarm (and took appropriate action) |

TABLE 3
Examples of vibration patterns used as feedback for
pump injection devices provided by electronic design.

| Action | Vibration Pattern |
|---|---|
| Dose Dialing | No action |
| Bolus complete | Two short pulses |
| Battery Status | Three short, three long, three short |
| Critical | (SOS) or alternatively continuous |
| | vibrations optionally short pauses. |
| Cartridge is empty or | Three short, Three long, Three short |
| close to empty (less | (SOS) |
| than approximately 3%) | or two long pulses with short pause |
| Infusion channel | Signals with a frequency of 400-650 |
| (needle) is blocked, | Hz periodically interrupted at 60 |
| or stalling, ie | i.p.m. (for example on for 0.5 s, |
| mechanism is blocked | off for 0.5 s). However as long as |
| | user presses button. |
| Infusion channel | Three short, three long, three short |
| (needle) leaks | (SOS) or triplet of short pulses and |
| | long pause; needs user confirmation |
| | to indicate that the user has |
| | understood the alarm (and took |
| | appropriate action) |

For example, the characteristics of the haptic signal enable a differentiation between a low life battery and a critical condition related to the medicament. The haptic signal can be generated by a haptic element including at least one of a vibrating motor, a vibrating battery, and a speaker with vibrating membranes of can include: (i) long vibrations (for example, about few seconds) with short humanly perceptible pauses (for example, about few milliseconds), (ii) short vibrations (for example, less than about 1 second) with long pause (for example, about few seconds), (iii) pauses with increasing durations, (iv) pauses with decreasing durations, (v) single/double/triple short vibrations; (vi) soft (for example, low pressure) vibration, (vii) strong (for example, high pressure) vibration. In some implementations, short (for example, less than about 3 seconds) haptic signals with simple haptic patterns (for example, with a single constant frequency or a pair of low and high frequencies) can be used to provide positive feedback. In some implementations, long (for example, longer than about 3 seconds) haptic signals with simple haptic patterns (for example, with a single constant frequency or a pair of low and high frequencies) or complex haptic patterns (for example, a combination of multiple frequencies or strong signals with high frequencies) can be used to provide an indication of a critical condition.

In some implementations, in response to successful transmission of the haptic signal, the medical device can reset the critical function (510) or can initiate a sleep mode to conserve the energy of the power source. In some implementations, the medical device is configured to periodically restart the process based on a preset time interval.

The features described can be implemented in digital electronic circuitry, or in computer hardware, firmware, software, or in combinations of them. The apparatus can be implemented in a computer program product tangibly embodied in an information carrier, for example, in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, for example, a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other implementations are within the scope of the following claims.

A number of implementations of the present disclosure have been described. Nevertheless, it will be understood that various modifications can be made without departing from the spirit and scope of the present disclosure. Accordingly, other implementations are within the scope of the following claims.

### LIST OF REFERENCE NUMBERS

100 exploded view of injection device
102, 200 injection devices
104 housing
106, 206, 304 medicament reservoir (cartridge)
108, 208 plunger
108a plunger rod
108b plunger head
109, 209 stopper
110, 210 injection button
111 electric drive
112 dosage knob
114 dosage window
115 tubular wall
116a distal end of injection device
116b proximal end of injection device
118 threading interface
120a, 120b, 120c, 220 haptic elements
122 needle
123 needle hub
124 handle
126 needle cap
128 outer needle cap
130 cap of injection device
132, 232 piston rod
134, 138, 234, 238 shafts
134a, 138a, 234a, 238a, 308a clutches
136, 236 motors
140, 240 unbalanced disk
142 detection module
143 housing
144, 244, 344 power sources
145 basket
146, 246, 346, 446 sensors
147 magnet
148, 348, 448 processors
149 spider
150 voice coil
152 diaphragm
154 suspension
202 cartridge holder
204 needle hub/tip
212 display
214 control panel
216 speaker
218 battery
222 power port
230 slits
242 belt drive
244 network interface
300 supplementary device
304 housing of supplementary device
310 housing of injection device
312 buttons
320 haptic element
331 display unit
322 input button
324 communications buttons
326 confirm buttons
330 eccentric mass counter weight
332 ball race bearing
334 front cap
342 detection module
348 processor
400 health monitor device
402 housing
404 display unit
406 input buttons
410 housing
412 base member
414 plates
416 piezoelectric elements
418 connection member
420 haptic element
422 amplifying part
424 circuit board
442 detection module
444 power source

1.-15. (canceled)

16. A medical handheld device comprising:
an actuating feature configured to generate a trigger signal;
a sensor configured to detect a functionality of the medical handheld device in response to the trigger signal;
a processor configured to determine a status of the medical handheld device; and
a haptic source configured to generate a haptic signal comprising information associated with the status of the medical handheld device.

17. The medical handheld device of claim 16, wherein the haptic source comprises a vibrating element.

18. The medical handheld device of claim 17, wherein the vibrating element comprises at least one of a vibrating motor, a vibrating battery, and a speaker with vibrating membranes.

19. The medical handheld device of claim 16, wherein the haptic source is enclosed within the medical handheld device.

20. The medical handheld device of claim 19, wherein the haptic source is coupled to a housing, wherein the housing comprises a material configured to enhance transmission of the haptic signal.

21. The medical handheld device of claim 20, wherein the haptic source is integrated into an activation button.

22. The medical handheld device of claim 16, further comprising a second sensor configured to detect a physiologic parameter of a patient.

23. The medical handheld device of claim 22, wherein the actuating feature is configured to generate the trigger signal in response to detecting the physiologic parameter.

24. The medical handheld device of claim 16, wherein the haptic signal comprises a pattern associated with the status of the medical handheld device.

25. The medical handheld device of claim 24, wherein the pattern comprises a combination of a frequency and a duration of the haptic signal.

26. The medical handheld device of claim 16, wherein the haptic source is included in an electric drive.

27. The medical handheld device of claim 26, wherein at least a portion of the electric drive is attached to a plunger rod that is configured to be displaced to expel a medicament.

28. The medical handheld device of claim 27, wherein the haptic source is integrated into at least one of an injection button, an input button, a dosage knob, and a dial grip.

29. The medical handheld device of claim 24, further comprising a motor comprising a first shaft configured to expel a medicament and a second shaft configured to generate the haptic signal.

30. A medical system comprising:
a medical handheld device comprising:
an actuating feature configured to generate a trigger signal,
a sensor configured to detect a functionality of the medical handheld device in response to the trigger signal,
a processor configured to determine a status of the medical handheld device, and
a haptic source configured to generate a haptic signal comprising information associated with the status of the medical handheld device; and
an external device comprising a component monitored by the medical handheld device.

31. The medical system of claim 30, wherein the haptic source comprises a vibrating element.

32. The medical system of claim 31, wherein the vibrating element comprises at least one of a vibrating motor, a vibrating battery, and a speaker with vibrating membranes.

33. The medical system of claim 30, wherein the haptic source is enclosed within the medical handheld device.

34. The medical system of claim 33, wherein the haptic source is coupled to a housing, wherein the housing comprises a material configured to enhance transmission of the haptic signal.

35. The medical system of claim 34, wherein the haptic source is integrated into an activation button.
